(19) United States
(12) Patent Application Publication
(10) Pub. No.: US 2013/0004997 A1
(43) Pub. Date: Jan. 3, 2013

(54) METHOD FOR PRODUCING SACCHARIFIED SOLUTION

(75) Inventor: Shigenobu Mitsuzawa, Saitama (JP)

(73) Assignee: HONDA MOTOR CO., LTD., Tokyo (JP)

(21) Appl. No.: 13/579,884

(22) PCT Filed: Mar. 4, 2011
(86) PCT No.: PCT/JP2011/055095

§ 371 Date: Aug. 17, 2012

(30) Foreign Application Priority Data
Mar. 19, 2010 (JP) 2010-064499
Jan. 14, 2011 (JP) 2011-005184

(51) Int. Cl. C12P 19/14

(52) U.S. Cl. 435/99

(57) ABSTRACT

A method for producing a saccharified solution is provided, by which a saccharide recovered as a saccharified solution can be increased. The saccharified solution is obtained by treating a substrate solution containing lignocellulosic biomass as a substrate with a saccharifying enzyme produced by a microorganism to prepare a substrate/saccharifying enzyme mixture liquid, and removing a residue of the substrate from the substrate/saccharifying enzyme mixture liquid. The concentration of the substrate in the substrate/saccharifying enzyme mixture liquid is adjusted to be in the range of 15 to 30% by mass. In the removal of the residue of the substrate, a saccharide adsorbed on the residue is extracted.

### TECHNICAL FIELD

The present invention relates to a method for producing a saccharified solution.

### BACKGROUND ART

From a viewpoint of prevention of global warming, reduction of the volume of carbon dioxide emission which is believed to be one of the causes thereof has been required recently. To this end, use of a blend fuel of a liquid hydrocarbon such as gasoline and ethanol for an automobile fuel has been studied. As such ethanol, ethanol produced by fermentation of plant substances, e.g. farm products, such as sugarcane and corn, can be used. Since plants themselves, which are source materials of the plant substances, have absorbed carbon dioxide by photosynthesis, when ethanol originated from the plant substances are burned, the amount of emitted carbon dioxide is equal to the amount of the carbon dioxide having been absorbed by the plants themselves. In other words, the so-called carbon-neutral effect can be obtained, such that the overall emission amount of carbon dioxide becomes zero in theory.

On the other hand, there is a drawback that large scale consumption of the sugarcane or corn as a source material for ethanol would reduce the amount of food supply.

Consequently, a technique for producing ethanol using nonfood biomass containing cellulose as the plant substances instead of sugarcane, corn, etc. has been studied. An example of the biomass containing cellulose is lignocellulosic biomass, including wood, rice straw, wheat straw, bagasse, bamboo, pulp or waste therefrom such as wastepaper.

In producing ethanol from the lignocellulosic biomass, the lignocellulosic biomass is used as a substrate; a saccharifying enzyme is added to the substrate to prepare an aqueous solution containing a saccharifying enzyme (a substrate/saccharifying enzyme mixture liquid); and cellulose and hemicellulose included in the substrate are degraded by the action of the saccharifying enzyme. As the saccharifying enzyme, those produced by microorganisms belonging to, for example, the genus Acremonium or the genus Trichoderma, are used.

Next, from the treated solution containing the degraded cellulose and hemicellulose, a residue of the substrate (lignocellulosic biomass) (hereinafter occasionally referred to as “biomass residue”) is removed to recover a saccharified solution. Then, an ethanol fermentation microorganism is added to the saccharified solution for ethanol fermentation to yield an ethanol aqueous solution. The yielded ethanol aqueous solution is subjected to a dehydration treatment by distillation, etc. to be purified finally to an ethanol fuel.

As the method for producing the saccharified solution is known a method, in which a saccharifying enzyme produced by an Acremonium cellulolyticus C1 strain is added to a substrate solution containing wastepaper as a substrate (e.g., see Patent Literature 1). According to the method for producing a saccharified solution using wastepaper as a substrate, a mixture liquid containing the substrate and the saccharifying enzyme is treated in a pH range of 2 to 8 to a saccharified solution. In this case the substrate/saccharifying enzyme mixture liquid contains 2.5 g of wastepaper in 50 milliliter and the substrate concentration is about 5% by mass.

As another method for producing the saccharified solution is known a method, in which a commercially available saccharifying enzyme is added to a substrate composed of, for example, rice straw for treating the same (e.g., see Patent Literature 2). According to the method for producing a saccharified solution by mating the substrate composed of rice straw with an added commercially available saccharifying enzyme, the substrate is treated with a saccharifying enzyme aqueous solution to a saccharified solution. In this case the substrate/saccharifying enzyme mixture liquid contains 50 mg of the substrate in 1 milliliter, and the substrate concentration is about 5% by mass.

### CITATION LIST

### Patent Literature

Patent Literature 1: Japanese Patent No. 4025848 (Paragraph [0024])

Patent Literature 2: Japanese Unexamined Patent Publication No. 2010-35431 (Paragraph [0063])

### SUMMARY OF INVENTION

### Technical Problem

When ethanol fermentation is carried out with a saccharified solution produced by the conventional method for producing a saccharified solution, it is preferable that a saccharide recovered as the saccharified solution should be as much as possible. Consequently, it is desirable that the substrate concentration in the substrate solution should be as high as possible.

However, there is a drawback that, if the substrate concentration is simply increased in the substrate solution, when a residue of the substrate is removed after the treatment with the saccharifying enzyme, the amount of a saccharide adsorbed on the residue to be removed together increases, and the amount of a saccharide to be recovered from the substrate/saccharifying enzyme mixture liquid as the saccharified solution decreases.

An object of the present invention is to provide a method for producing a saccharified solution, eliminating the above drawback, able to increase the recovery rate of a saccharide as the saccharified solution with respect to a substrate/saccharifying enzyme mixture liquid.

### Solution to Problem

To attain the object, the present invention is characterized by a method for producing a saccharified solution, by treating a substrate solution containing lignocellulosic biomass as a substrate with a saccharifying enzyme produced by a microorganism to prepare a substrate/saccharifying enzyme mixture liquid, and removing a residue of the substrate from the substrate/saccharifying enzyme mixture liquid to obtain a saccharified solution; wherein the concentration of the substrate in the substrate/saccharifying enzyme mixture liquid is prepared to be in the range of 15 to 30% by mass, and in the removal of the residue of the substrate from the substrate/saccharifying enzyme mixture liquid, a saccharide adsorbed on the residue is extracted.

According to the method for producing a saccharified solution of the present invention, by adjusting the substrate concentration in the substrate/saccharifying enzyme mixture liquid to be in the above range, excessive amount of the residue derived from the lignocellulosic biomass as the substrate can be prevented. Owing to this, the amount of a saccharide adsorbed on the residue becomes not excessive, and more saccharide can be recovered as the saccharified solution with respect to the substrate/saccharifying enzyme mixture liquid.

If the substrate concentration is less than 15% by mass, the efficiency is compromised, because the amount of the lignocellulosic biomass as a substrate is small and the amount of a saccharide itself to be yielded by the action of the saccharifying enzyme is small. If the substrate concentration exceeds 30% by mass, a residue derived from the substrate increases, and the amount of a saccharide adsorbed on the residue increases too, so that the amount of a saccharide to be recovered as the saccharified solution decreases.

Next, according to a method for producing a saccharified solution of the present invention, when a residue of the substrate is removed from the substrate/saccharifying enzyme mixture liquid, a saccharide adsorbed on the residue is extracted. As a result, more saccharide can be recovered as the saccharified solution.

According to a method for producing a saccharified solution of the present invention, the substrate concentration in the substrate solution is adjusted to be in the range, and when a residue of the substrate is removed from the substrate/saccharifying enzyme mixture liquid, a saccharide adsorbed on the residue is extracted, and consequently the saccharide concentration in the recovered saccharified solution can be in a range of 6 to 18% by mass.

Lignocellulosic biomass to be used as the substrate has a structure, in which lignin is bound to cellulose or hemicellulose. Therefore, the action of the saccharifying enzyme on the cellulose or hemicellulose is retarded by lignin. Under such circumstances, according to a method for producing a saccharified solution of the present invention, the lignocellulosic biomass is preferably treated with ammonia or steam for removing lignin bound to cellulose or hemicellulose to prepare a substrate solution, which is then treated with the saccharifying enzyme to obtain the substrate/saccharifying enzyme mixture liquid. The substrate solution can be easily saccharified by the saccharifying enzyme, once the lignin bound to cellulose or hemicellulose in the lignocellulosic biomass is removed.

Further, according to a method for producing a saccharified solution of the present invention, the treatment of the substrate solution by the saccharifying enzyme is preferably carried out by adding the saccharifying enzyme in an amount ranging from 1.2 to 70.2 U in terms of microcrystalline cellulose degradation activity and from 0.8 to 46.8 U in terms of xylan degradation activity with respect to 1 g of the substrate/saccharifying enzyme mixture liquid. If the amount of the saccharifying enzyme to be added is less than 1.2 U in terms of the microcrystalline cellulose degradation activity, or less than 0.8 U in terms of the xylan degradation activity with respect to 1 g of the substrate/saccharifying enzyme mixture liquid, the lignocellulosic biomass may occasionally not be adequately saccharified. Even if the amount of the saccharifying enzyme to be added exceeds 70.2 U in terms of the microcrystalline cellulose degradation activity or 46.8 U in terms of the xylan degradation activity with respect to 1 g of the substrate/saccharifying enzyme mixture liquid, more effect can be hardly obtained, and an increase of the production cost cannot be suppressed.

### BRIEF DESCRIPTION OF DRAWINGS

FIG. 1 is a graph showing the relationship between the substrate concentration in the substrate/saccharifying enzyme mixture liquid with a 1st saccharifying enzyme and the recovery rate of the yielded saccharified solution with respect to the total amount of the substrate/saccharifying enzyme mixture liquid.

FIG. 2 is a graph showing the relationship between the substrate concentration in the substrate/saccharifying enzyme mixture liquid with the 1st saccharifying enzyme and the saccharide concentration in the yielded saccharified solution.

FIG. 3 is a graph showing the relationship between the substrate concentration in the substrate/saccharifying enzyme mixture liquid with the 1st saccharifying enzyme and the recovery rate of a saccharide yielded in the saccharified solution with respect to the total amount of the substrate/saccharifying enzyme mixture liquid.

FIG. 4 is a graph showing the relationship between the substrate concentration in the substrate/saccharifying enzyme mixture liquid with a 2nd saccharifying enzyme and the recovery rate of the yielded saccharified solution with respect to the total amount of the substrate/saccharifying enzyme mixture liquid.

FIG. 5 is a graph showing the relationship between the substrate concentration in the substrate/saccharifying enzyme mixture liquid with the 2nd saccharifying enzyme and the saccharide concentration in the yielded saccharified solution.

FIG. 6 is a graph showing the relationship between the substrate concentration in the substrate/saccharifying enzyme mixture liquid with the 2nd saccharifying enzyme and the recovery rate of a saccharide yielded in the saccharified solution with respect to the total amount of the substrate/saccharifying enzyme mixture liquid.

### DESCRIPTION OF EMBODIMENTS

An embodiment of the present invention will be described in more detail referring to the appended drawings.

According to a method for producing a saccharified solution in the current embodiment, rice straw as lignocellulosic biomass, which is a substrate, is treated with 25 mass/volume-% ammonia water for removing lignin bound to cellulose or hemicellulose in the rice straw to prepare a substrate solution. In the treatment for removing lignin with the ammonia water, for example, the substrate solution is kept at a temperature of 80° C. for 8 hours for a reaction.

As the result of the treatment with the ammonia water, the pH of the substrate solution becomes in a range of 13 to 14.

Therefore, an acid selected out of sulfuric acid, hydrochloric acid, nitric acid, acetic acid, citric acid, phosphoric acid, etc. is further added to the substrate solution to adjust the pH of the same to be in the range of 3 to 7. The acids may be used singly, or in combinations of two or more.

Then a saccharifying enzyme aqueous solution is added to the substrate solution with the pH adjusted to be in the range of 3 to 7. As the saccharifying enzyme, GC220 (trade name, by Genencor, a division of Danisco US Inc.), or Acremonium (trade name, by Meiji Seika Kaisha, Ltd.) is favorably utilized, however any other saccharifying enzyme can substitute. The amount of the saccharifying enzyme to be added is preferably in a range of 1.2 to 70.2 U in terms of microcrystalline cellulose degradation activity and in a range of 0.8 to 46.8 U in terms of xylan degradation activity with respect to 1 g of the substrate/saccharifying enzyme mixture liquid, more preferably in a range of 5.9 to 46.8 U in terms of microcrystalline cellulose degradation activity and in a range of 3.9 to 31.2 U in terms of xylan degradation activity, and for example 23.4 U in terms of microcrystalline cellulose degradation activity and 15.6 U in terms of xylan degradation activity. The amount of the saccharifying enzyme to be added reduced to the concentration with respect to the total amount of the substrate/saccharifying enzyme mixture liquid is preferably in a range of 1 to 60% by mass, more preferably in a range of 5 to 40% by mass, and, for example, at 20% by mass.

Microcrystalline cellulose degradation activity can be measured as follows. To a 50 μL-sample of an aqueous solution containing a saccharifying enzyme, 350 μL of a 200 mM acetate buffer solution (pH 4) and 400 μL of a 4 mass/volume-% microcrystalline cellulose (Grade No. 1.02331.0500, by Merck & Co., Inc.) suspension are added, and the mixture is left reacting with stirring at 50° C. for 15 min. Then, 800 μL of an aqueous solution containing 30 mass/volume-% Rochelle salt, 1 mass/volume-% dinitrosalicylic acid, and 1.6 mass/volume-% sodium hydroxide is added thereto, and the mixture is centrifuged at 15000×g for 20 min. The absorbance for the light with the wavelength of 540 nm of a solution that is obtained by treating the supernatant at 100° C. for 5 min is measured and the concentration of the dissolved reducing sugars is calculated using glucose as a standard substance. In this regard, art enzyme amount releasing 1 micromole of a reducing sugar per 1 min is defined as 1 U.

Similarly, xylose degradation activity can be measured as follows. First, a 1 mass/volume-% xylan (originated from birchwood, by Sigma-Aldrich Japan K.K.) suspension is boiled for 2 hours, thereafter centrifuged at 10000×g for 20 min, and the supernatant is freeze-dried as a soluble xylan and used as the substrate for xylose degradation activity. Next, to a 20 μL-sample of an aqueous solution containing a saccharifying enzyme, 180 μL of a 200 mM acetate buffer solution (pH 4) and 200 μL of a 1 mass/volume-% soluble xylan aqueous solution are added and the mixture is left reacting with stirring at 50° C. for 15 min. Then, 800 μL of an aqueous solution containing 30 mass/volume-% Rochelle salt, 1 mass/volume-% dinitrosalicylic acid, and 1.6 mass/volume-% sodium hydroxide is added thereto. The absorbance for the light with the wavelength of 540 nm of a solution that is obtained by treating at 100° C. for 5 min is measured and the concentration of the dissolved reducing sugars is calculated using xylose as a standard substance. In this regard, an enzyme amount releasing 1 micromole of a reducing sugar per 1 min is defined as 1 U.

Next, the substrate solution with the added saccharifying enzyme is kept in a temperature range of 30 to 50° C., e.g. at 50° C., for a time range of 50 to 150 hours, e.g. for 72 hours to degrade and saccharify cellulose or hemicellulose in the rice straw as a substrate by the action of the saccharifying enzyme. In this case 60 to 85% by mass of the cellulose or hemicellulose in the rice straw as a substrate is degraded and saccharified. As a result, a substrate/saccharifying enzyme mixture liquid containing saccharides, such as glucose, xylose, and arabinose, can be obtained.

The substrate/saccharifying enzyme mixture liquid after saccharification contains biomass residue generated as the result of degradation of the cellulose or hemicellulose in the rice straw as a substrate. Therefore, next, the biomass residue is separated and removed from the substrate/saccharifying enzyme mixture liquid to recover the saccharified solution.

In this regard, according to the method for producing a saccharified solution of the current embodiment, by adjusting the concentration of the rice straw as a substrate in the substrate solution to be in the range of 15 to 30% by mass, excessive biomass residue can be prevented.

Further, according to a method for producing a saccharified solution of the current embodiment, when the biomass residue is separated and removed from the substrate/saccharifying enzyme mixture liquid, a saccharide adsorbed on the biomass residue is extracted. Examples of a method for separating and removing the biomass residue as well as extracting a saccharide adsorbed on the biomass residue include methods using centrifugation, filtration, a filter press, a vacuum dehydrator, a belt press dehydrator, a screw press dehydrator, and a multi-disk dehydrator.

According to the method for producing a saccharified solution of the current embodiment, more saccharide with respect to the substrate/saccharifying enzyme mixture liquid can be recovered as the saccharified solution, by adjusting the concentration of the rice straw as a substrate in the substrate solution to be in the range of 15 to 30% by mass and extracting the saccharide adsorbed on the biomass residue. As a result, the saccharified solution contains a saccharide usable for fermentation, such as glucose, xylose, and arabinose, at a concentration in a range of, for example, 6 to 18% by mass.

Next, the recovery rate of a saccharide yielded, when the concentration of the rice straw as a substrate in the substrate solution was varied in a range of 10 to 35% by mass, and the saccharide adsorbed on the biomass residue was extracted, was measured as follows. First, to a specific amount of rice straw, 25 mass/volume-% ammonia water was added, and the mixture was kept at a temperature of 80° C. to left reacting for 8 hours, thereby to remove lignin bound to cellulose or hemicellulose in the rice straw. Then, sulfuric acid was added to the substrate solution containing the rice straw from which lignin is removed to adjust the pH to about 4.

Then a saccharifying enzyme aqueous solution was added to the substrate solution adjusted to about pH 4 and mixed to the final content of the substrate from 20 to 30% by mass, and the saccharifying enzyme aqueous solution at 20% by mass with respect to the total amount of the substrate/saccharifying enzyme mixture liquid. Then, a saccharification treatment was conducted by keeping the substrate/saccharifying enzyme mixture liquid at a temperature of 50° C. for 72 hours. As for the saccharifying enzyme, GC220 (by Genencor, a division of Danisco US Inc.) as the 1st saccharifying enzyme, or Acremonium (by Meiji Seika Kaisha, Ltd.) as the 2nd saccharifying enzyme was used. After the saccharification treatment, the biomass residue was separated and removed by centrifugation (8000×g, 20 min) to recover a saccharified solution.

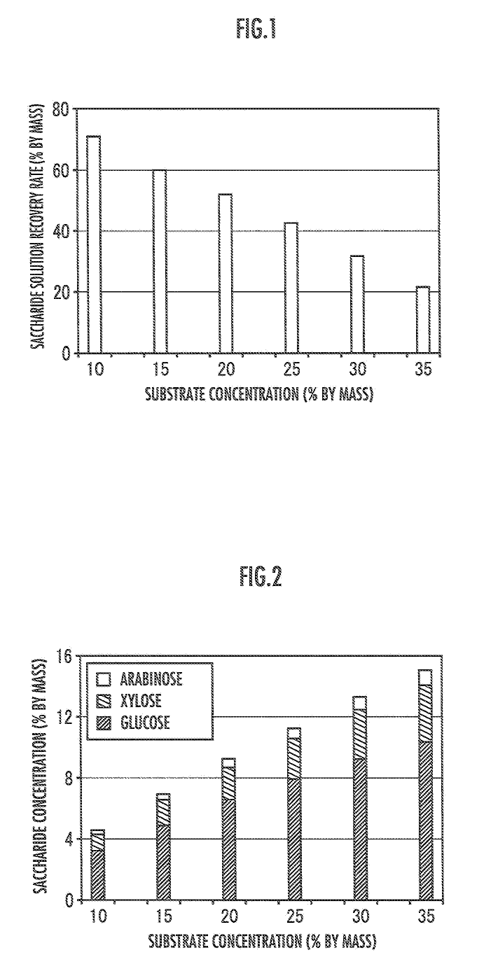

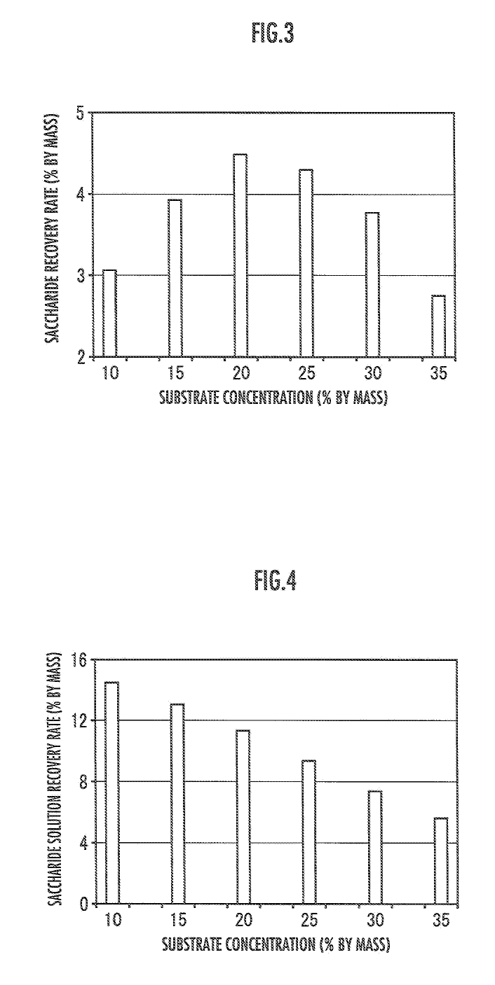

The recovery rate (% by mass) of the yielded saccharified solution with respect to the total amount of the substrate/saccharifying enzyme mixture liquid corresponding to each substrate concentration, in the event GC220 (by Genencor, a division of Danisco US Inc.) was used as a saccharifying enzyme is shown in FIG. 1. The saccharide concentration (% by mass) in the then yielded saccharified solution corresponding to each substrate concentration is shown in FIG. 2. Further, the recovery rate of a saccharide yielded in each saccharified solution with respect to the total amount of the substrate/saccharifying enzyme mixture liquid corresponding to each substrate concentration is shown in FIG. 3.

The saccharide recovery rate (% by mass) at each substrate concentration in FIG. 3 is calculated by multiplying the saccharide concentration (% by mass) at each substrate concentration in FIG. 1 and the saccharide solution recovery rate (% by mass) at each substrate concentration in FIG. 2 followed by division by 100.

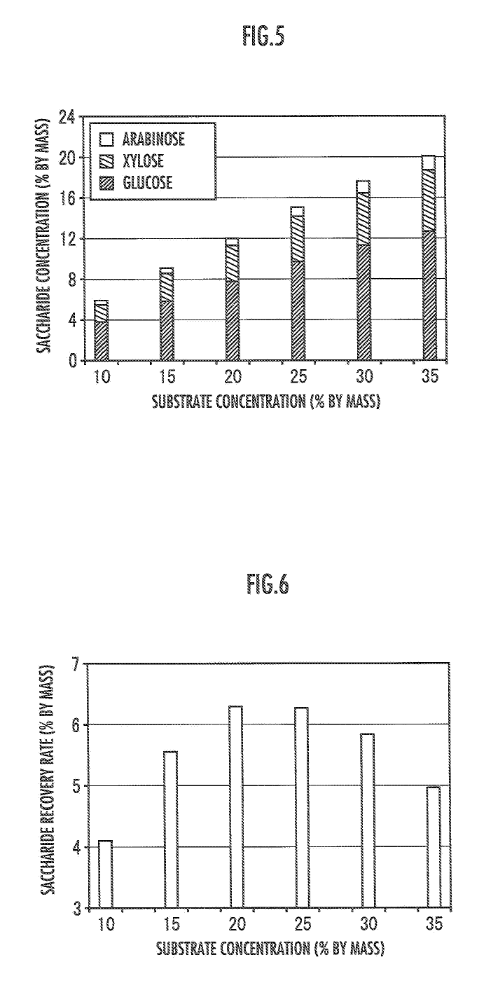

The recovery rate (% by mass) of the yielded saccharified solution with respect to the total amount of the substrate/saccharifying enzyme mixture liquid corresponding to each substrate concentration, in the event Acremonium (by Meiji Seika Kaisha, Ltd.) is used as a saccharifying enzyme is shown in FIG. 4. The saccharide concentration (% by mass) in the then yielded saccharified solution corresponding to each substrate concentration is shown in FIG. 5. Further, the recovery rate of a saccharide yielded in each saccharified solution with respect to the total amount of the substrate/saccharifying enzyme mixture liquid corresponding to each substrate concentration is shown in FIG. 6.

The saccharide recovery rate (% by mass) at each substrate concentration in FIG. 6 is calculated by multiplying the saccharide concentration (% by mass) at each substrate concentration in FIG. 4 and the saccharide solution recovery rate (% by mass) at each substrate concentration in FIG. 5 followed by division by 100.

From FIG. 3 and FIG. 6, it is obvious that, according to the method for producing a saccharified solution of the current embodiment, when the concentration of the rice straw as a substrate in the substrate solution is adjusted to be in the range of 15 to 30% by mass, and the saccharide adsorbed on the biomass residue is extracted, more saccharide can be recovered than in the case where the concentration of the rice straw is outside the range.

1. A method for producing a saccharified solution, by treating a substrate solution, which is prepared by treating lignocellulosic biomass with ammonia or steam for removing lignin bound to cellulose or hemicellulose, with a saccharifying enzyme produced by a microorganism to prepare a substrate/saccharifying enzyme mixture liquid, and removing a residue of the substrate from the substrate/saccharifying enzyme mixture liquid to obtain a saccharified solution;
wherein upon preparing the substrate/saccharifying enzyme solution mixture liquid, the saccharifying enzyme is added to give a concentration range of 1 to 60% by mass with respect to the total amount of the substrate/saccharifying enzyme mixture liquid,
the concentration of the substrate in the substrate/saccharifying enzyme mixture liquid is adjusted to be in the range of 15 to 30% by mass,
in the removal of the residue of the substrate from the substrate/saccharifying enzyme mixture liquid, a saccharide adsorbed on the residue is extracted using any one method selected from the group consisting of methods by any of a filter press, a vacuum dehydrator, a belt press dehydrator, a screw press dehydrator and a multi-disk dehydrator, and
the concentration of the saccharide in the saccharified solution is in a range of 6 to 18% by mass.

2. (canceled)

3. (canceled)

4. The method for producing a saccharified solution according to claim 1, wherein the treatment of the substrate solution with the saccharifying enzyme is carried out by adding the saccharifying enzyme in an amount ranging from 1.2 to 70.2 U in terms of microcrystalline cellulose degradation activity and from 0.8 to 46.8 U in terms of xylan degradation activity with respect to 1 g of the substrate/saccharifying enzyme mixture liquid.

5. The method for producing a saccharified solution according to claim 1, wherein the treatment of the substrate solution with the saccharifying enzyme is carried out by adding the saccharifying enzyme in an amount ranging from 5.9 to 46.8 U in terms of microcrystalline cellulose degradation activity and from 3.9 to 31.2 U in terms of xylan degradation activity with respect to 1 g of the substrate/saccharifying enzyme mixture liquid.

6. (canceled)

7. The method for producing a saccharified solution according to claim 1, wherein the treatment of the substrate solution with the saccharifying enzyme is carried out by adding the saccharifying enzyme to give a concentration range of 5 to 40% by mass with respect to the total amount of the substrate/saccharifying enzyme mixture liquid.

8. The method for producing a saccharified solution according to claim 1, wherein the substrate solution containing the added saccharifying enzyme is kept in a temperature range of 30 to 50° C. for a time range of 50 to 150 hours to degrade and saccharify cellulose or hemicellulose in the substrate by the action of the saccharifying enzyme.

9. (canceled)

10. The method for producing a saccharified solution according to claim 1, wherein the saccharified solution contains, as a saccharide, glucose, xylose, or arabinose.
